(19) United States
(12) Patent Application Publication
(10) Pub. No.: US 2004/0147434 A1
(43) Pub. Date: Jul. 29, 2004

(54) Use of enzyme inhibitors of the dipeptidypeptidase iv (ec3.3.14.5) in addition to the aminopeptidase n (ec 3.4.11.2), individually or in a combination thereof, and pharmaceutical preparations thereof for the prevention and/or therapy of ischaemia-caused acute and chronic neurodegenerative process and illnesses, for example

(76) Inventors: Siegfried Ansorge, Hohenwartie (DE); Uwe Lendeckel, Magdeburg (DE); Frank Striggow, Gerwisch (DE); Klaus Neubert, Halle (DE); Klaus Reymann, Niederndodeleben (DE); Thilo Kahne, Magdeburg (DE)

Correspondence Address: HODGSON RUSS LLP, ONE M & T PLAZA, SUITE 2000, BUFFALO, NY, 14203-2391, US

(21) Appl. No.: 10/250,475

(22) PCT Filed: Dec. 21, 2001
(86) PCT No.: PCT/EP01/15196

(30) Foreign Application Priority Data
Jan. 2, 2001 (DE) 101 00 053.7

(51) Int. Cl. A61K 38/17; A61K 38/10; A61K 38/08; A61K 38/06; A61K 31/69

(52) U.S. Cl. 514/12; 514/13; 514/14; 514/15; 514/16; 514/17; 514/18; 514/19; 514/64

(57) ABSTRACT

The present invention relates to a process wherein, by a separate or simultaneous administration and effect of inhibitors of the enzymes alanyl aminopeptidase (APN) and dipeptidyl peptidase IV (DP IV) or of enzymes having the same or a similar substrate specifity, the damage of cerebral tissue as a consequence of an ischemia or a cranial/cerebral trauma can be reduced or prevented.

The invention shows that the separate or combined application of inhibitory substances of the above-mentioned enzymes or of corresponding preparations or administration forms is definitely suitable for a prevention and therapy of the above indications.

The present invention describes the reduction of cerebral damage processes conditional on ischemia by the inhibitory action of the enzymatic activity of amino peptidase N (APN; E.C. 3.4.11.2.; CD 13) and/or dipeptidyl peptidase IV (DP IV, E.C. 3.4.14.5.; CD 26) and of enzymes of the same or similar substrate specifity, respectively. A neuroprotective effect is achieved by the combined application, and by the separate application as well, of respective specific inhibitors of said enzymes on the basis of amino acid derivatives, peptides or peptide derivatives.

The interruption of the blood supply to cells, tissues or organs is called ischemia. Such a situation is critical in particular in those cases where a continuous supply of oxygen and/or nourishing substances (e. g. glucose) is necessary. This is applicable particularly to the central nervous system (CNS), since nerve cells, specifically, react extremely sensitively to an interruption of the supply with oxygen and glucose. Even a short-term ischemia, for example as a consequence of a stroke or of a cardiac infarction, results into a neuronal cell death in the cerebral areas involved. In modern industrial countries, a cerebral ischemia is the most frequent cause of mortality and invalidity. In Germany, the incidence rate of stroke per year is approximately 400 per 100,000 inhabitants. The cellular mechanisms of damaging processes caused by ischemia have many aspects and are understood unsufficiently up to now due to their complexity. Thus, measures directed to a prevention and therapy are problematical [Dimagl, U, ladecola, C, Moskowitz, M A (1999), Pathobiology of ischemic stroke: an integrated view; Trends Neurosci 22: 391-399].

It is known that membrane-located peptidases as, for example, DP IV or APN play a key role in the process of the activation and clonal expression of immune cells, particularly of T lymphocytes [Fleischer B: CD26, a surface protease involved in T cell activation. Immunology Today 1994; 15:180-184; Lendeckel U et al.: Role of alanyl aminopeptidase in growth and function of human T cells. International Journal of Molecular Medicine 1999; 4:17-27; Riemann D et al.: CD13—not just a marker in leukemia typing. Immunology Today 1999; 20: 83-88]. Several functions of mitogen-stimulated mononuclear cells (MNC) or accumulated T lymphocytes as, for example, DNA synthesis, production and secretion of immune-stimulating cytokines (IL-2, IL-6, IL-12, IFN-γ) and helper functions for B cells (synthesis of IgG and IgM) may be inhibited in the presence of specific inhibitors of DP IV and APN [Schön E et al.: The dipeptidyl peptidase IV, a membrane enzyme involved in the proliferation of T lymphocytes. Biomed. Biochim. Acta 1985; 2: K9-K15; Schön E et al.: The role of dipeptidyl peptidase IV in human T lymphocyte activation. Inhibitors and antibodies against dipeptidyl peptidase IV suppress lymphocyte proliferation and immunoglobulin synthesis in vitro. Eur. J. Immunol. 1987; 17: 1821-1826; Reinhold D et al.: Inhibitors of dipeptidyl peptidase IV induce secretion of transforming growth factor β1 in PWM-stimulated PBMNC and T cells. Immunology 1997; 91: 354-360; Lendeckel U et al. : Induction of the membrane alanyl aminopeptidase gene and surface expression in human T cells by mitogenic activation. Biochem. J. 1996; 319: 817-823; Kähne T et al.: Dipeptidyl peptidase IV: A cell surface peptidase involved in regulating T cell growth (Review). Int. J. Mol. Med. 1999; 4: 3-15; Lendeckel U et al.: Role of alanyl aminopeptidase in growth and function of human T cells (Review). Int. J. Mol. Med. 1999; 4: 17-27].

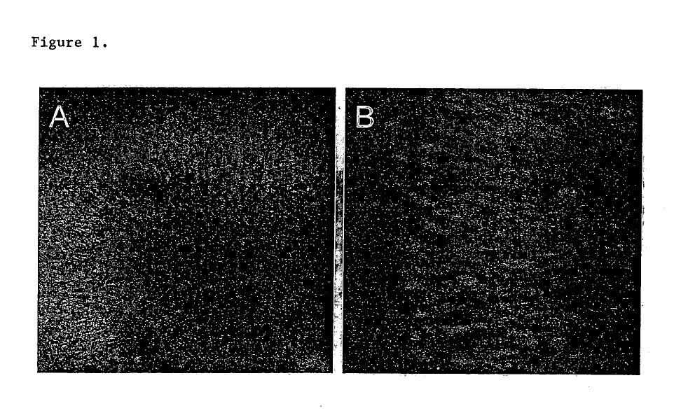

Within the CNS, DP IV and APN are localized on distinguished types of cells and in different areas (see below): Recently, a DP IV localized in the intracellular space in the cytosol could be detected [Gilmartin L and O'Cuinn G: Neurosci Res 1999; 34:1-11]. Possibly, this enzyme is identical to the protein labeled in hippocampal pyramidal cells for the first time by our working group (see FIG. 1, fluorescence photograph hipocampus, I-63/labelled with fluorescein). Particularly those nervous cells are known for their outstanding importance for processes of learning and memory [Bliss TVP, Collingridge GL (1993) A synaptic model of memory: Long-term potentiation in the hippocampus. Nature 361: 31-39] and also for their marked sensitivity against ischemic events [Striggow, F, Riek M, Breder J, Henrich-Noak P, Reymann K G, Reiser G (2000). The protease thrombin is an endogenous mediator of hippocampal neuroprotection against ischemia at low concentrations but causes degeneration at high concentrations. Proc. Natl. Acad. Sci. (USA) 97: 2264-2269].

Peripheric immune cells, e. g. T cells, are not present in the CNS normally. In pathologic situations, they may, however, enter the CNS, particularly in cases where the blood/brain barrier becomes permeable. Exactly such a situation occurrs with cerebral ischemia [Fischer S, Clauss M, Wiesnet M, Renz, D, Schaper W, Karliczek G F (1999), Hypoxia induces permeability in brain microvessel endothelial cells via VEGF and NO. Am J Physiol 276: C812-C820]. In addition, APN is an essential component of the cell membrane of pericytes. Particularly on pericytes, expression and activity of the APN decisively determine the functionality of the blood/brain barrier, and an impairment of the functionality of the blood/brain barrier is accompanied by a change of the APN expression [Kunz J et al.: J Neurochem. 1994; 62: 2375-2386; Kunz J et al.. : J Neuroimmunol 1995; 59: 41-55; Ramsauer M et al: J Cereb Blood Flow Metab 1998; 18: 1270-1281; Alliot F et al.: J Neuroscience Research 1999; 58: 367-378].

Both enzymes dipeptidyl peptidase IV (DP IV) and also alanyl aminopeptidase (APN) are expressed in several areas of the cerebrum. Soluble and membrane-bound DP IV was detected in the cerebrum of the rat [Alba F et al.; Peptides 1995; 16: 325-329]. In the same way, DP IV could be detected in the cytosol of cerebra of guinea pigs [Gilmartin L and O'Cuinn G: Neurosci Res 1999; 34:1-11]. Besides the well-documented presence of DP IV on the endothelium of microvessels of the cerebrum, the enzyme was localized on Schwann cell membranes at their contact points to axons of sensoric nerve ends in several mammals [Dubovy P and Malinowsky L: Histochemical J 1984; 16: 473-475] as well as at the luminal membrane of ependymal cells in the ventricles and in the central channel of the spinal cord [Bourne et al.: Biochem J 1989; 259: 69-80]. In addition, DP IV was detected unequivocally on the endothelium of the microvasculatory of the pig cerebrum and, there, also on cells of the striatum [Barnes K et al.; Eur J Neurosci 1994; 6: 531-537].

The expression of APN in the CNS is effected on a broader cellular and spatial basis. The neuronal cell lines H4, SK-N-SH, the oligodendrocyte line MO3.13 and the astrocyte lines GL-15, U-87MG and U-373 MG distinguish by a surface expression of APN [Lachance C et al.: J Virology 1998; 72: 6511-6519]. This APN surface expression apparently is causative for the neurotropism of the human corona virus 229E [Lachance C et-al.: J Virology 1998; 72: 6511-6519]. In addition to the expression on the vessel endotheliurn, APN is also found on astrioglia cells and pericytes [Barnes K et al.: Eur J Neurosci 1994; 6: 531-537].

Facing the background that inhibitors of APN and DP IV effect—as can be proved—a production and release of the immunosuppressive cytokine “transforming growth factor β1” (TGF-β1) from immune cells [Reinhold D et al.; Immunology 1997; 91: 354-360; Lendeckel U et al.; Int J Molecular Medicine 1999; 4: 17-27, Kähne T. et al.; Int J Molecular Medicine 1999; 4: 1-15] the fact may be noteworthy that TGF-β1 apparently has a great importance for a limitation of damages of the CNS. These clearly neuroprotective functions of TGF-β1 could be detected at ischemic and excitoxic lesions and after a neurotrauma as well [Ruocco A et al: Cereb Blood Flow Metab 1999; 19: 1345-1353; Yamashita K et al.; Brain Res 1999; 836: 139-145; Docagne F et al.; FASEB J 1999; 13: 1315-1324; Morganti-Kossmann M C et al.; J Neurotrauma 1999; 16: 617-628; Ho T W et al.; Exp Neurol 2000; 161: 664-675; Henrich-Noack P et al.; Stroke 1996; 27: 1609-1615; Lehrmann E et al.; Exp Neurol 1995; 131: 114-123; Knuckey N W et al.; Brain Res Mol Brain Res 1996; 40: 1-14; Zhu Y et al.; Brain Res 2000; 866: 286-298]. TGF-β1 is expressed in the whole brain, comparatively more at the location of damages, where astrocytes and microglia cells produce and release TGF-β1 primarily [O'Keefe G M et al.; Eur J Immunol 1999; 29: 1275-1285; Docagne F et al.; FASEB J 1999; 13: 1315-1324].

The invention described here is based on the surprising finding that a combined and separate effect of enzyme inhibitors of DP IV (E.C. 3.4.14.5.) and of APN (E.C. 3.4.11.2.) significantly improves the survival of neuronal cells in cultivated hippocampus slices (prepared from iuvenile rat cerebra) after an ischemia. This was not known up to now. Thus, our invention shows that a combined and simultaneous administration of the above-mentioned inhibitors and of corresponding compositions and administration forms, respectively, is definitely suitable for a prevention and/or therapy of cerebral damage processes induced by an ischemia.

Searches in our laboratories up to now showed that the DNA synthesis and proliferation of human immune cells, for example of peripheric mononuclear cells (MNC) or of T lymphocytes, is inhibited by the simultaneous and separate administration of inhibitors of DP IV (e. g. Lys[Z(NO₂)] thiazolidide=I49) and of APN (e. g. actinonine), and the generation and secretion of zytokines is changed [Reinhold D et al; Inhibitors of dipeptidyl peptidase IV induce secretion of transforming growth factor β1 in PWM-stimulated PBMNC and T cells. Immunology 1997; 91: 354-360]. Whether an antiproliferative effect of inhibitors of DP IV and APN is also the basis of the neuroprotective effects of both substance classes described here, remains still unclear, but is probable.

The application of inhibitors of the enzymes DP IV and/or APN is a novel method and supplementary therapy form for the above-mentioned diseases.

The inhibitors of DP IV and of APN applied in accordance with the present invention may be applied in the form of pharmaceutically applicable formulation complexes as inhibitors, substrates, pseudo-substrates, peptides having inhibitory effect and peptide derivatives as well as antibodies for this enzyme.

Preferred effectors for DP IV are, for example, Xaa-Pro dipeptides, corresponding derivatives, preferably dipeptide phosphonic acid diaryl esters and their salts, Xaa-Xaa-(Trp)-Pro-(Xaa)ₙ peptides (n=0 to 10), corresponding derivatives and their salts, or amino acid-(Xaa) amides, corresponding derivatives and their salts, wherein Xaa is an α-amino acid or -imino acid or an α-amino acid derivative or -imino acid derivative, respectively, preferably N^{ε}-4-nitrobenzyloxycarb-onyl-L-lysine, L-proline, L-tryptophane, L-isoleucine, L-valine, and cyclic amines as, for example pyrrolidine, piperidine, thiazolidine, and their derivatives serve as the amide structure. Such compounds and their preparation were described in an earlier patent (K. Neubert at al., D D 296 075 A5).

Preferred inhibitors of alanyl aminopeptidase are bestatin (Ubenimex), actinonine, probestine, phebestine, RB3014 or leuhistine.

The inhibitors are administered simultaneously or separately with known carrier substances. The administration may occur, on the one hand, in the form of a topical application by means of cremes, ointments, pastes, gels, solutions, sprays, liposomes, shaken mixtures, hydrocolloid dressings and other dermatologic bases/vehicles including instillative application and, on the other hand, in the form of a systemic application for an oral, transdermal, intravenous, subcutaneous, intracutaneous or intramuscular application in suitable formulations or in a suitable galenic form.

### WORKING EXAMPLES

### Example 1

Neuronal Localization of DP IV (or of an Enzyme Similar to DP IV) in the Hippocampus

FIG. 1: Confocal fluorescence photograph of a hippocampus slice after loading with an irreversible and fluorescence-labeled inhibitor of DP IV: Hippocampus slices were incubated with a I63/fluorescein conjugate (10 μM) for 60 min. In the course of this time, I63/fluorescein irreversibly binds to DP IV, whereby said enzyme is fluorescence-labeled. Subsequently, the slices were washed with phosphate buffer 3 times for 20 min in order to remove inhibitor (including fluorescence) which was not bound. DP IV-positive neurons could be observed thereafter in all CA regions of the hippocampus and in the area dentata as well. FIG. 1A shows the DP IV labeling in the CA1, CA2 and CA3 regions of a hippocampal slice. In FIG. 1B, there are shown DP IV-positive pyramide cells within the CA1 region. Both fluorescence photographs were taken with a confocal laser scanning microscope (Zeiss LSM 510, objective 10× (A) or 20× (B)).

### Example 2

Neuroprotective Effect Against Experimentally Caused Ischemia by Incubation With Synthetic Inhibitors of DP IV and APN

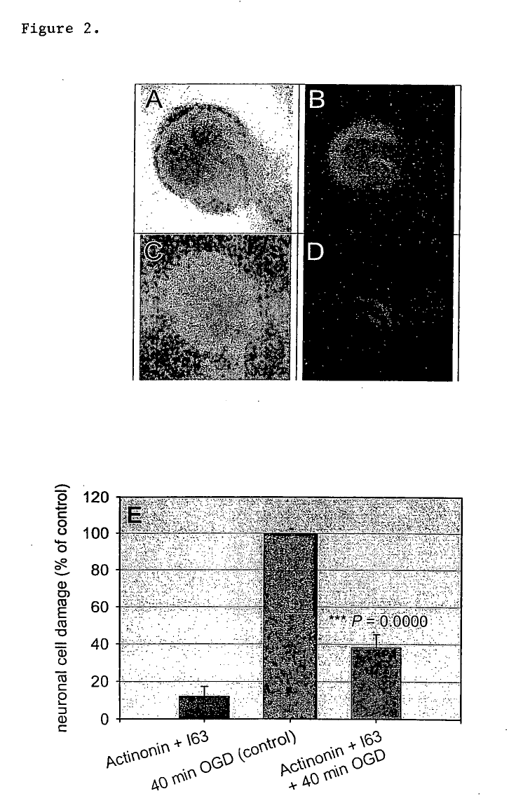

FIG. 2: Protective effect of inhibitors of DP IV (I63) and APN (actinonine) on the survival of hippocampal neurons after a transient deprivation of oxygen and glucose: There are shown in the FIG. transmission photographs (respective left sides) and fluorescence photographs (respective right sides) of hippocampus slices 24 h after an experimental ischemia. The intensity of the red fluorescence corresponds to the neuronal cell damage (B, D). The slices shown in FIGS. 2A and 2B were subjected to a 40 min deprivation of oxygen and glucose in the absence of DP IV or APN inhibitors. In contrast thereto, the slices shown in FIGS. 2C and 2D were incubated with I63 (1 μm; DP IV inhibitor) and with actinonine (10 μm; APN inhibitor) 24 h before the ischemia, during the ischemia and 24 h after the ischemia. The evaluation of n=8 experiments each including at least 10 slices per condition is shown in FIG. 2E: There are shown the average values±standard deviation. The protective effect occurring at a simultaneous application of I63 and actinonine is significant (P=0,0000; calculated by means of a two side heteroscedastic t test) and is about 62%.

Organotypical hippocampus slices were prepared and cultivated according to Striggow F et al. (Striggow, F, Riek M, Breder J, Henrich-Noak P, Reymann K G, Reiser G (2000). The protease thrombin is an endogenous mediator of hippocampal neuroprotection against ischemia at low concentrations but causes degeneration at high concentrations. Proc. Natl. Acad. Sci. (USA) 97: 2264-2269). After 12 days in culture, a cerebral ischemia was simulated by experiment by a 40 min deprivation of oxygen and glucose. In order to achieve this, glucose (10 mM) in the media was replaced by mannit-ol (10 mM), and the media was supplied with a gas consisting of 95% N₂/5% CO₂ instead of 95% O₂/5% CO₂. After further 24 h under normal culture conditions, all slices were incubated with propidium iodide (PI) for 1 h. PI enters only such cells the cell membrane of which is damaged [Macklis J D, Madison R D (1990); Progressive incorporation of propidium iodide in cultured mouse neurons correlates with declining electrophysiological status: a fluorescence scale of membrane integrity. J Neurosci Methods 31: 43-46]. Only after said time, there occurrs a characteristic red fluorescence, the intensity of which is proportional to the neuronal cell damage. The fluorescence photographs were taken by means of an inverse fluorescence microscope (Nikon Diaphot, objective: 4×) and a CCD camera (Visitron systems) and were evaluated subsequently (Nikon Lucia M software package).

### Example 3

Protective Effect of a DP IV Inhibitor in the Absence of Inhibitors of APN

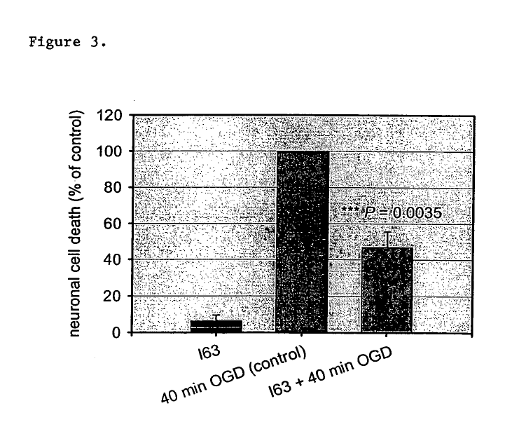

FIG. 3: Effect of inhibitors of DP IV (I63) on the survival of hippocampal neurons after a transient deprivation of oxygen and glucose: The experiments were carried out as described in connection with FIG. 2, with the only exception that exclusively I63 (1 μm, inhibitor of DP IV) was applied 24 h before, during and 24 h after the ischemia. There are shown the average values±standard deviation determined from n=4 experiments. The protective effect is significant (P=0,0035; calculated by means of a two side heteroscedastic t test) and is about 53%.

### Example 4

Protective Effect of an APN Inhibitor in the Absence of Inhibitors of DP IV

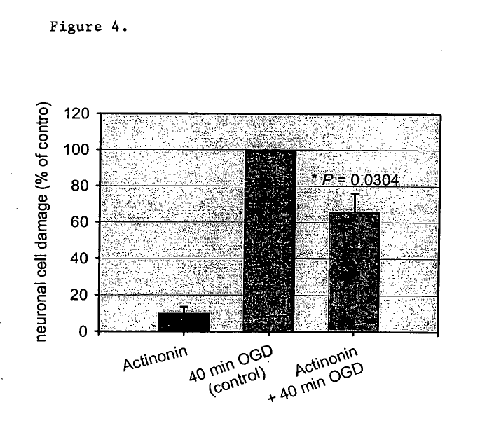

FIG. 4: Effect of inhibitors of APN (actinonine) on the survival of hippocampal neurons after a transient ischemia caused by experiments: The experiments were carried out as described in connection with FIG. 2, with the only exception that exclusively actinonine (10 μm, inhibitor of APN) was applied 24 h before, during and 24 h after the ischemia. There are shown the average values±standard deviation determined from n=5 experiments. The protective effect is significant (P=0,0304; calculated by means of a two side heteroscedastic t test) and is about 34%.

1. Use of inhibitors of dipeptidyl peptidase IV (DP IV) and of enzymes having the same or a similar substrate specifity (DP IV-analogous enzyme activity) separately or in any combination with inhibitors of alanyl aminopeptidase (aminopeptidase N, APN) and of enzymes having the same or a similar substrate specifity (APN analogous enzyme activity) for a prevention and therapy of damages, induced by an ischemia, in the central nervous system, independent of the fact whether such processes are acute or chronic processes and independent of the fact whether the application is an application to a human or an application to an animal.

2. Use according to claim 1, wherein the inhibitors of DP IV are Xaa-Pro-dipeptides (Xaa=α-amino acid and side chain protected derivative, respectively), corresponding derivatives, preferably dipeptide phosphonic acid diaryl esters, dipeptide boronic acids (e. g. Pro-boro-Pro) and their salts, Xaa-Xaa-(Trp)-Pro-(Xaa)ₙ peptides (Xaa=α-amino acid, n=0 to 10), corresponding derivatives and their salts and amino acid-(Xaa)-amides, respectively, corresponding derivatives and their salts, wherein Xaa is an α-amino acid or a side chain protected derivative, respectively, preferably N^{ε}-4-nitrobenzyloxycarbonyl-L-lysine, L-proline, L-tryptophane, L-isoleucine, L-valine, and cyclic amines as, for example, pyrrolidine, piperidine, thiazolidine, and their derivatives serve as the amide structure.

3. Use according to claim 1, wherein amino acid amides, e. g. N^{ε}-4-nitrobenzyl-oxycarbonyl-L-lysine thiazolidide, pyrrolidide and piperidide as well as the corresponding 2-cyanothiazolidide, 2-cyanopyrrolidide and 2-cyanopiperidide derivatives are used as DP IV inhibitors.

4. Use according to claim 1, wherein, as the inhibitors of APN, actinonine, bestatine, leuhistine, phebestine, amastatine, probestine, β-aminothiols, α-aminophos-phinic acids, α-aminophosphinic acid derivatives, preferably D-Phe-ψ[PO(OH)—CH₂]-Phe-Phe and their salts, are used.

5. Use of inhibitors or of inhibitor combinations according to any of the claims 1 to 4 for a prevention and therapy of cerebral damages caused by an ischemia, preferably of ischemic or hemorrhagic stroke, after a cranial/cerebral trauma, after a heart standstill, after a cardiac infarction or as a consequence of heart surgical operations (e. g. bypass surgeries).

6. Pharmaceutical preparations, comprising inhibitors of dipeptidyl peptidase IV (DP IV) and of enzymes having a DP IV-analogous enzyme activity in combination with inhibitors of one of the enzymes alanyl aminopeptidase (aminopeptidase N, APN) and of enzymes having the same or a similar substrate specifity (APN analogous enzyme activity) and in combination with per se known carrier, additive and/or auxiliary substances.

7. Pharmaceutical preparation according to claim 6, comprising, as inhibitors of DP IV preferably Xaa-Pro dipeptides (Xaa=α-amino acid and side chain protected derivative, respectively), corresponding derivatives, preferably dipeptide phosphonic acid diaryl esters, dipeptide boronic acids (e. g. Pro-boro-Pro) and their salts, Xaa-Xaa-(Trp)-Pro-(Xaa)ₙ peptides (Xaa=α-amino acid, n=0 to 10), corresponding derivatives and their salts and amino acid-(Xaa) amides, corresponding derivatives and their salts, respectively, wherein Xaa is an α-amino acid or a side chain protected derivative respectively, preferably N^{ε}-4-nitrobenzyloxycarbonyl-L-lysine, L-proline, L-tryptophane, L-isoleucine, L-valine, and cyclic amines as, for example, pyrrolidine, piperidine, thiazolidine, and their derivatives serve as the amide structure.

8. Pharmaceutical preparation according to claim 6, comprising, as inhibitors of DP IV preferably amino acid amides, e. g. N^{ε}-4-nitrobenzyloxycarbonyl-L-lysine-thiazolidide, pyrrolidide and piperidide as well as the corresponding 2-cyanothiazolidide, 2-cyanopyrrolidide and 2-cyanopiperidide derivatives.

9. Pharmaceutical preparation according to claim 6, comprising as inhibitors of APN, actinonine, leuhistine, phebestine, amastatine, bestatine, probestine, β-aminothiols, α-aminophosphinic acids, α-aminophosphinic acid derivatives, preferably D-Phe-ψ[PO(OH)—CH₂]-Phe-Phe and their salts.

10. Pharmaceutical preparation according to any of the claims 6 to 9, comprising two or more inhibitors of DP IV or of enzymes of DP IV-analogous enzymatic activity, of APN or of enzymes having APN-analogous enzymatic activity, in a compartimentally separate formulation in combination with per se known carrier, auxiliary and/or additive substances for a simultaneous or immediately timely consecutive administration with the aim of a combined effect.

11. Pharmaceutical preparation according to any of the claims 6 to 10 for the systemic application for an oral, transdermal, intravenous, subcutaneous, intracutaneous, intramuscular, rectal, vaginal, sublingual administration together with per se known carrier, auxiliary and/or additive substances.

12. Pharmaceutical preparations according to any of the claims 6 to 10 for the topical application in the form of, for example, creams, ointments, pastes, gels, solutions, sprays, liposomes, shaken mixtures, hydro-colloid dressings and other dermatological bases/vehicles including instillative applications.
